(19) United States
(12) Patent Application Publication
(10) Pub. No.: US 2007/0238696 A1
(43) Pub. Date: Oct. 11, 2007

(54) Dosage unit for cardioprotection

(76) Inventors: Herbert M. Dean, Waban, MA (US); Robert E. Weinstein, Boston, MA (US); Allan M. Weinstein, Potomac, MD (US)

Correspondence Address: MESMER & DELEAULT, PLLC, 41 BROOK STREET, MANCHESTER, NH, 03104, US

(21) Appl. No.: 11/748,061

(22) Filed: May 14, 2007

Related U.S. Application Data
(62) Division of application No. 10/076,937, filed on Feb. 15, 2002.
Application No. 10/076,937 is a continuation of application No. 09/717,987, filed on Nov. 21, 2000, now abandoned.

(51) Int. Cl. A61K 31/44; A61K 31/50; A61K 31/70; A61P 9/04

(52) U.S. Cl. 514/52; 514/249; 514/347

(57) ABSTRACT

A method for secondary cardiovascular prevention in a patient includes formulating a single dosage unit containing a beta-adrenergic blocking agent and a cholesterol-lowering agent that is a statin where the single dosage unit contains a quantity of medications sufficient for secondary prevention of a heart attack in the patient, and administering the single dosage unit to the patient for secondary prevention of a heart attack. The method further includes the secondary prevention of a heart attack in a non-hypertensive patient.

This application is a Divisional application of Ser. No. 10/076,937, filed on Feb. 15, 2002, which is Continuation application of Ser. No. 09/717,987, filed on Nov. 21, 2000, now abandoned.

### BACKGROUND OF THE INVENTION

1. Field of the Invention

The present invention relates to treatments for reducing the risk of cardiovascular disease. Particularly, the present invention relates to combinations of agents that antagonize beta-adrenergic function and agents that reduce cholesterol. More particularly, the present invention relates to beta-blocker agents and agents to reduce cholesterol for the treatment of cardiovascular disease and for the purpose of reducing medication error and increasing therapeutic compliance.

2. Description of the Prior Art

Cardiovascular risk can be lowered both by agents that antagonize beta-adrenergic function and agents that reduce cholesterol. Despite the abundance of presently marketed medications, no medicinal formulation is presently available which provides a user with an agent to antagonize beta-adrenergic function and an agent to lower cholesterol in a single dosage unit. Such a dosage unit would simplify treatment, increase convenience, reduce cost, and enhance compliance.

In this discussion, the term “cardiovascular disease” is intended to refer to coronary heart disease (CDH), and also include strokes and peripheral vascular disorders. Cardiovascular disease is responsible for about 40% of the deaths in industrialized countries. More than 500,000 Americans die from heart disease each year, the leading cause of death in the U.S. The American Heart Association estimates that the total annual cost of medical care and lost productivity due to heart disease is $12 billion to $24 billion. Annually, 1.5 million Americans suffer a heart attack, and people who have had a heart attack are at high risk of having another one.

The pathophysiology leading to atheromatous plaque formation associated with cardiovascular disease is complex. Efforts at prevention of cardiovascular disease include lifestyle efforts and medicinal interventions. Examples of lifestyle efforts include dietary measures to control weight, elimination of smoking and appropriate exercise.

Two types of agents are known to reduce morbidity and mortality from these diseases. The agents are adrenergic blocking agents and cholesterol reducing agents. The efficacy of each is acknowledged for primary prevention, i.e. individuals who are not known to have cardiovascular disease but are at risk, and secondary prevention, i.e. individuals who are known to have cardiovascular disease, and are at risk for progression and further events.

Cholesterol is an important factor in the pathogenesis and atheromatous plaque formation of cardiovascular disease. Total cholesterol level measured in the blood, which can be done in a non-fasting state, has been shown to correlate predictably with CHD over a large range of values. Low-density lipoprotein (LPL) cholesterol constitutes approximately 65% of total cholesterol and is currently the primary target for measuring risk and monitoring response. The current guidelines of the National Cholesterol Education Program consider an LDL cholesterol of more than 160 mg/dl elevated, a value of 130-150 mg/dl borderline high, and a value of less than 130 mg/dl desirable for primary prevention. A value of under 100 mg/dl is now considered desirable for secondary prevention in individuals with a previous coronary event. Such a level is considered optimal with respect to preventing CHD. High-density lipoprotein (HDL) cholesterol is another component of total cholesterol. Higher levels of HDL are considered protective. While medications are presently available to effect a reduction in LDL, methods and agents to raise HDL levels are presently not as established.

Based on clinical trial data, a 10% reduction in total cholesterol levels reduces lifetime risk for CHD by greater than 50% if the reduction occurs before age 40, 39% if by age 50, and drops to 27% at age 60. This indicates the need to identify individuals at an early age to reduce cardiovascular events. Reduction of LDL and its surrogate, total cholesterol, typically involves dietary measures to control weight, dietary fat, saturated fat, and dietary cholesterol intake. Most adults will additionally require intervention with medication, and the most effective class of agents is hydroxymethyl glutaryl coenzyme A (HMG-CoA) reductase inhibitors known as “statins.”

Numerous studies have demonstrated the benefit of statins in reducing CHD. The Scandinavian Simvastin Survival Study demonstrated a 42% reduction in deaths from CHD. Similar results have been demonstrated with pravastatin in reducing non-fatal as well as fatal coronary events. Large studies have consistently demonstrated that reduction of cholesterol with statins is effective in secondary prevention in individuals with known disease as well as in primary prevention for individuals who are not known to have heart disease. Meta-analysis of five CHD studies has shown drug therapy with a combination of diet and a statin can decrease disease progression by 50% and increase regression threefold.

The following statins are presently available in the United States: atorvastatin, cervastatin, pravastatin, fluvastatin, lovastatin, and simvastatin. It is reasonable to expect the development of others. These drugs are typically administered once per day in the evening, but lovastatin can be given with either the morning or evening meal. Maximal plasma LDL reduction varies from 25% with fluvastatin to 60% with atorvastatin. Reduction of plasma triglyceride, and modest elevation of HDL may additionally be achieved with these agents.

The economic benefit of statin treatment is reflected by a study in which 20 mg of lovastatin was administered to individuals with serum cholesterol of over 250 mg/dl. The economic benefit of statin treatment is estimated to achieve a cost-effectiveness ratio of under $20,000 per year of life saved in men and women of all ages.

Another category of agent commonly utilized, as a preventative measure in treating cardiovascular disease are the beta-adrenergic blocking agents. Examples of such agents listed in the current Physicians Desk Reference (PDR 2000) include propanolol, atenolol, timolol maleate, carteolol, penbutolol, nadolol, acebutolol hydrochloride, and metaprolol succinate. Indications for these agents include treatments for hypertension, angina pectoris due to coronary atherosclerosis, cardiac arrythmias, and reduction of cardiovascular mortality in patients who have survived the acute phase of myocardial infarction.

Large studies indicate that ten of thousands of lives could be saved each year if more people were utilizing a beta-blocker after having a heart attack. One study done at the University of Maryland reviewed medical records of more than 200,000 people who had suffered a heart attack, 34% of whom received beta-blockers. During the next two years, people treated with beta-blockers had a 40% lower mortality rate compared to those who had not. Another significant report from Yale University disclosed that one year after a heart attack, patients over 65 years of age who had not taken beta blockers were 14% less likely to be alive than those who had taken them.

Decreased compliance is known to occur when multiple medications are used, and individuals with cardiovascular disorders are known to commonly utilize many medications. The problems of achieving compliance include the inconvenience of taking multiple dosage units over a long period of time and confusion with multiple medication particularly in older individuals, the age group in which these cardio-preventative medications are typically required. Simplification is a desired goal, and many of these problems can be ameliorated by incorporating the desired beta-adrenergic blocking agents and cholesterol lowering agents into a single dosage unit.

Further, cost factors as well as outcomes are now being carefully considered, and improvements provided by this invention can save medical personnel expenditures. Cost of providing one dosage unit is less than cost of providing many. For example, managed care organizations typically pay a pharmacy filling cost for each prescription filled to reimburse for the time and personnel needed to fill each prescription. Costs and filling efforts for more than a single prescription would be saved by this invention. Successful prophylactic therapy is clearly preferable and less costly, compared to treatment for symptomatic disease, prolonged illness, and/or disability, which require expensive medical resources including clinic visits, hospitalizations, and major cardiovascular surgery.

Therefore, what is needed is a device and method that combines agents that antagonize beta-adrenergic function and agents that reduce cholesterol. What is further needed is a device and method that includes the administration of a single dose.

### SUMMARY OF THE INVENTION

It is an objective of the present invention to provide a single oral cardiovascular protective medicinal formulation comprising a beta-adrenergic antagonist and a cholesterol-lowering agent. It is another objective of the present invention to provide a method for enhancing compliance with effective measures for preventing cardiovascular disease by providing an oral formulation comprising a beta-adrenergic antagonist and a cholesterol lowering agent, instructing and administering this formulation to a patient in need thereof. It is a further objective to provide such medications in accordance with scientific evidence of therapeutic efficacy.

The present invention contemplates an interventional measure that is neither within the scope of lay individuals nor presently available to lay individuals. The clear need for cardiovascular preventive treatment and the failure of patients to avail themselves of such treatment underscores the present need for the formulations of this invention. Combining these agents to provide a single dosage unit for a user would simplify treatment, increase convenience, reduce cost, and enhance compliance with use over the long-term required.

The present invention achieves these and other objectives by providing a system for the treatment of cardiovascular disease that requires a combined single dosage unit regimen and method for reducing medication error and enhancing therapeutic compliance of combined medication agents for treatment of such disease. The system includes a single dosage unit that combines at least an agent for antagonizing beta-adrenergic function and an agent for reducing cholesterol, and instructions for administering the single dosage unit. The single dosage unit may also contain one or more of folic acid, vitamin B6, vitamin B12, and vitamin E. The present invention also includes a method of reducing medication error and enhancing therapeutic compliance of combined agents for the treatment of cardiovascular disease. The method includes formulating in a single dosage unit a beta-adrenergic blocking agent and a cholesterol-lowering agent, and instructing the use of the single dosage unit for treating cardiovascular disease. The method also includes formulating in a single dosage unit one or more of folic acid, vitamin B6, vitamin B12, and vitamin E.

### DETAILED DESCRIPTION OF THE PREFERRED EMBODIMENT

The following detailed description of the invention is provided to aid those skilled in the art in practicing the present invention, however, it should not be construed to unduly limit the present invention. Variations and modifications in the disclosed embodiments may be made by those of ordinary skill in the art without departing from the scope of the present invention.

Compliance with medication is an important consideration in preventing or otherwise treating medical disorders. The simpler the medication regimen, the better the adherence over time. The present invention simplifies dosing of a plurality of medications for both primary as well as secondary prevention of cardiovascular disease by a single dosage formulation. The present invention simplifies dosing of a plurality of medications for both primary as well as secondary prevention of cardiovascular disease preferably using a dosage, once-a-day formulation. The present invention provides the components of a regimen for preventing cardiovascular disease in a convenient manner, compared to the current need to purchase or prescribe individual components.

The present invention provides a single dosage unit that incorporates a beta-adrenergic antagonist and an agent to lower cholesterol in accord with scientific evidence of their efficacy. Other agents may also be incorporated. Examples of desirable components include the vitamins B6, B12 and folic acid, essential nutritional cofactors in the metabolism of homocysteine. Homocysteine elevation is an independent risk factor in vascular disease and a five-year prospective study has shown that the risk of heart attack for individuals with elevated homocysteine levels is 3.4 fold greater in subjects with elevated homocysteine levels. In individuals with elevated homocysteine, lowering of levels usually responds to supplementation with folic acid. In some instances supplementation with vitamins B6 and B12 may also be necessary to lower homocysteine levels.

The inclusion of folic acid in formulations of the present invention in the range of about 200 mcg to about 2000 mcg is considered desirable. It is also desirable to include folic acid, along with B6 in the range of about 2 mg to about 300 mg, or B12 in the range of about 10 mcg to about 1000 mcg, or both, so as to assure normal homocysteine levels.

The naturally occurring antioxidant, vitamin E, is another example of an agent that is considered to prevent coronary artery disease and strokes and which is considered desirable for inclusion in formulations of the present invention. Epidemiological data has shown a reduction of cardiovascular risk with vitamin E supplementation of at least 100 IU/day. This benefit does not occur at lesser dosages such as a 30 IU/day replacement dosage typical of multivitamin use. In a study of 39,000 health professionals followed for four years, men with a median intake of 419 IU/day of vitamin E had a 44% relative risk reduction compared to men whose median intake was 6 IU/day.

The present invention anticipates that any or all of the active components of the dosage unit may be prepared for immediate release, or if desired, delayed release so as to alter rate of absorption. Materials and methods by which this may be accomplished are well known in the art, for example, by employing hydrophilic matrix materials such as methylcellulose, hydroxyethylcellulose, and hydroxypropylcellulose.

The present invention further anticipates formulations that require dosing schedules of more than once a day, although once-a-day dosing is preferred. The present invention also anticipates that formulations may be in tablet, capsule, caplet, syrup, liquid, or other dosage forms commonly employed for oral administration of medicaments.

The following are examples of proposed formulations of the present invention containing both a beta-adrenergic antagonist and a cholesterol-lowering agent:

### EXAMPLE 1

The synthetic beta1-selective adrenoreceptor blocking agent atenolol in a range from about 10 mg to about 100 mg combined with the cholesterol lowering agent atorvastatin in a range from about 10 mg to about 80 mg. A preferred formulation is a single dosage unit of 25 mg of atenolol combined with 20 mg of atorvastatin, preferably taken once a day.

### EXAMPLE 2

The formulation of Example 1 which further includes folic acid in a range of about 200 mcg to about 2000 mcg, vitamin B12 in a range of about 10 mcg to about 1000 mcg, vitamin B6 in a range of about 2 mg to about 300 mg, and vitamin E in a range of about 100 IU to about 800 IU.

### EXAMPLE 3

Propanalol hydrochloride 160 mg in a sustained release formulation suitable for once-per-day dosing combined with pravistatin in a range of about 10 mg to about 40 mg, the formulation is to be taken once-a-day at bedtime.

### EXAMPLE 4

The non-selective beta-adrenoreceptor blocking agent timolol maleate in the amount of 10 mg combined with lovastatin in a range of about 5 mg to about 40 mg. This formulation might be taken twice a day.

### EXAMPLE 5

The beta1-selective beta-adrenoreceptor blocking agent metoprolol tartrate in the amount of 100 mg combined with fluvastatin in a range of about 10 mg to about 40 mg. This formulation might be taken twice a day.

These examples are not meant to be inclusive and it is contemplated that, other dosages, other beta-blocking agents, and other cholesterol lowering agents for primary or secondary prevention of cardiovascular disease are within the scope of this invention.

Various modifications and alterations of the present invention may be appreciated based on a review of this disclosure, and such changes and additions are intended to be within the scope and spirit of this invention as defined by the following claims.

What is claimed is:

1. A method of secondary cardiovascular prevention in a patient, the method comprising:
formulating a single dosage unit containing a beta-adrenergic blocking agent and a cholesterol-lowering agent that is a statin wherein the single dosage unit contains a quantity of medications sufficient for secondary prevention of a heart attack in the patient; and
administering the single dosage unit to the patient for secondary prevention of a heart attack.

2. The method of claim 1 wherein the formulating step further includes adding one or more of folic acid, vitamin B6, and vitamin B12.

3. The method of claim 1 wherein the formulating step further includes formulating the single dosage unit as a once-a-day dosage unit.

4. A method for secondary cardiovascular prevention in a non-hypertensive patient, the method comprising:
formulating a single dosage unit containing a sufficient quantity of a beta-adrenergic blocking agent and a cholesterol-lowering agent that is a statin for secondary prevention of a heart attack in the non-hypertensive patient; and
administering the single dosage unit to the non-hypertensive patient for secondary prevention of a heart attack.

5. The method of claim 1 wherein the formulating step further includes adding one or more of folic acid, vitamin B6, and vitamin B12.

6. The method of claim 1 wherein the formulating step further includes formulating the single dosage unit as a once-a-day dosage unit.

7. A method for secondary cardiovascular prevention in a non-hypertensive patient, the method comprising:
formulating a single dosage unit consisting essentially of a sufficient quantity of a beta-adrenergic blocking agent and a cholesterol-lowering agent that is a statin for secondary prevention of a heart attack in the non-hypertensive patient; and
administering the single dosage unit to the non-hypertensive patient for secondary prevention of a heart attack.

8. A method of making a dosage unit for cardioprotection for secondary cardiovascular prevention in a non-hypertensive patient, the method comprising:
formulating a single dosage unit containing a sufficient quantity of a beta-adrenergic blocking agent and a cholesterol-lowering agent that is a statin for secondary prevention of a heart attack in the non-hypertensive patient.
